(19) United States
(12) Patent Application Publication
(10) Pub. No.: US 2020/0214578 A1
(43) Pub. Date: Jul. 9, 2020

(54) Cuff-Type Monitoring Device For Monitoring Cardiovascular Parameters

(71) Applicant: WITHINGS, Issy Les Moulineaux (FR)

(72) Inventors: Xianzhi ZHANG, Paris (FR); Jean-Louis WANG, Puteaux (FR); Virginie VISSAC, Plaisir (FR); Pierre-Antoine CUNIASSE, Bagneux (FR); Benoît TUCOULAT, Paris (FR); Mélanie ALSBERGHE-PATEL, Paris (FR)

(73) Assignee: WITHINGS, Issy Les Moulineaux (FR)

(21) Appl. No.: 16/733,850

(22) Filed: Jan. 3, 2020

(30) Foreign Application Priority Data
Jan. 4, 2019 (EP) 19305010.1

(51) Int. Cl. A61B 5/021; A61B 5/0416; A61B 5/0404; A61B 5/024

(52) U.S. Cl. CPC A61B 5/02141; A61B 5/0416; A61B 5/0404; A61B 2562/0204; A61B 2505/07; A61B 2560/045; A61B 2560/0468; A61B 5/02438

(57) ABSTRACT

A cuff-type monitoring device, for collecting cardiovascular data relating to an individual user, comprising a control unit housing including a pump, a cuff assembly including a resilient cuff holder and an interface member arranged at the control unit housing, provided integral with the control unit housing or as a separate part, wherein there is provided at least a pneumatic coupling and an electrical coupling through the interface member, for coupling the cuff assembly to the control unit housing, wherein the resilient cuff holder comprises two retaining members, the interface member comprising two complementary retaining members, configured to be snap-fit with the retaining members, and assembly method to assemble the device.

This application claims priority under the Paris Convention to European Patent Application No. 193305010.1 filed on Jan. 4, 2019.

### FIELD OF THE DISCLOSURE

The present disclosure relates to devices for analysing cardiovascular parameters of an individual. More particularly, it relates to a multifunction cuff-type monitoring device for monitoring cardiovascular parameters of such individual.

### BACKGROUND OF THE DISCLOSURE

Such device is configured to collect cardiovascular parameters like blood pressure and at least one additional signal, in particular an electrical signal representative of an electrocardiogram or an acoustic signal representative of a phonocardiogram. More specifically, there is provided an inflatable bladder to pressurize the arm of the patient. Further the monitoring device comprises on the one hand a cuff assembly configured, in use, to surround an arm of the individual, and on the other hand a control unit housing which forms together with the cuff assembly a single unit. Since the control unit housing is somewhat tubular, the link member between the cuff assembly and the control unit housing should be simple and mechanically robust. But it is necessary to provide various connections (electrical, pneumatic) between the cuff assembly and the control unit housing, which is somehow contradictory with the goal previously set out. The inventors have endeavored to find a smart solution to respond to various combined constraints.

### SUMMARY OF THE DISCLOSURE

According to one aspect of the present disclosure, it is disclosed a cuff-type monitoring device, for collecting cardiovascular data relating to an individual user, comprising:
a control unit housing including at least a pump,
a cuff assembly including a resilient cuff holder and configured, in use, to surround an upper limb of the individual, and
an interface arranged at the control unit housing, provided as a separate part from the control unit housing,
   wherein there is provided at least a pneumatic coupling and at least an electrical coupling through the interface member, for coupling the cuff assembly to the control unit housing,
   wherein the resilient cuff holder comprises two retaining members,
   the interface member comprising two complementary retaining members, configured to be snap-fit with the retaining members.

Thanks to these dispositions, the assembly of such device is very simple. The assembled device is aesthetic, since no pneumatic coupling and no electrical coupling is visible from the outside of the device.

In the present document, the geometric reference may be defined as follows for the interface member: the assembling direction is denoted R, the elongation axis of the interface member is denoted X, the transverse direction of the interface member is denoted T.

Besides, one or more of the following features can be used optionally, taken alone or in combination.

According to an option, the interface member is a separate part interposed between the control unit housing and the cuff assembly for securing them together. The interface member can be manufactured from a material different than the control unit housing. Each of these two parts can have differentiated constraints and mechanical requirements. Each of these two parts are manufactured and prepared independently from one another.

According to an alternate option, the interface member is made integral with the control unit housing. In this case (not illustrated at Figures) a 3D printing technique can be used to manufacture this part, this technique allows to obtain complex 3D shapes.

According to an option, the interface member is screwed to the control unit housing. Screwing is well mastered and forms a reliable mechanical solution for fastening function.

The interface member may comprise a frustoconical thru bore for each screw. Each of such thru bore is designed to receive a countersunk head screw that will not protrude from a face of the interface member. This assembly is therefore flush and is thus not aggressive for the cuff assembly. There may be provided 2 or 4 screws in the attachment.

According to a particular option, the interface member exhibits a symmetry with regard to plane X,R. Thereby the interface member is rather simple to manufacture.

According to a particular option, the interface member exhibits a symmetry with regard to a median plane Tm (cf FIG. 6). It prevents the risk of mistake when assembling the interface member.

According to a particular option, the interface member exhibits a double symmetry with regard to plane Tm and with regard to plane X,R. Manufacturing of such item is therefore simplified.

According to a particular option, the cuff holder can be a monolithic resilient part.

According to an option, the retaining members may be formed as hooks. Such hooks protrude from the cuff holder baseline layer. There may be provided a large footprint for these hooks, namely a foot/shoe larger than the hook arm profile itself, thereby providing a strong implant on the cuff holder sheet; large disassembly torque can thus be withstood.

According to an option, there is provided two hooks spaced from one another of a distance (E3) at least 5 cm or at least 50% of the height of the cuff assembly. Here the height is taken along the elongation axis of the interface member X. The assembly can thus withstand large disassembly torque.

According to an option, there is provided interlock members (138) received in holes along a transverse direction T. The interlock members prevent disassembly of complementary retaining members versus retaining members.

According to an option, the interface member comprises transverse holes (168) for receiving the interlock members (138), said transverse holes having in some examples a diameter less than 2 mm. Advantageously, there is no screw visible outside the device; the interface member can be a thin, elegant part.

According to an option, the interlock elements (138) can be formed as interlock pins. According to another option, the interlock elements (138) can be formed as interlock thin screws, for example, rod-like screw with sotted head.

According to an option, the interface member (6) exhibits a first face (6A) oriented towards the control unit housing (7) and a second face (6B) oriented towards the cuff assembly, wherein the first face is concave and is configured to follow the cylindrical body of the control unit housing. Therefore, the contact area between the interface member and the control unit housing is maximized and the mechanical attachment is therefore made stronger.

According to an option, the second face is substantially flat. The contact of the cuff assembly is smooth and not aggressive.

According to an option, both faces may also be concave, in particular when the interface member (6) may have a large transverse dimension, the second face (6B) may exhibit a slight concavity to follow the concavity of the cuff holder.

According to an option, the cuff holder is a resilient plastic part with retaining members or hooks integrally formed therein. Such part is easy to manufacture and assembly is simplified.

According to an option, the interface member is an elongated flat part, plastic part, in some examples with a thickness (6T) along assembling direction R less than 6 mm. Thereby, with such a thin interface member, the cuff-type monitoring device exhibit an aesthetic appearance and is a compact solution. The length of the interface member along the elongation axis is in some examples at least 10 cm.

According to an option, there is provided in a middle portion of the interface member at least a through hole for electrical wire connection/coupling, and a least one through hole for pneumatic fluid piping/coupling. Not only one multi-wire connection is possible but also two pneumatic fluid connections are provided internally to the interface member, i.e. through holes arranged in the interface member.

According to an option, there is provided an ECG electrode (32) disposed around at least a part of the control unit housing (7) and the interface member (6) is configured to securely fix the ECG electrode on the control unit housing. Advantageously, the interface member has an additional electrical function.

The present disclosure is also directed to an assembly method for assembling a cuff-type monitoring device, for collecting cardiovascular data relating to an individual user, the method comprising:
Providing a control unit housing (7) including at least a pump,
Providing a cuff assembly (10) including a resilient cuff holder (3) and
Providing an interface member (6)
Fixing the interface member to the control unit housing,
Assembling the cuff assembly to the interface member, by inserting two retaining members into two complementary retaining members,
Securing the cuff assembly to the interface member by inserting interlock elements (138).

According to an option, the method may further comprise:
disassembling the cuff assembly from the interface member, by lifting the intermediate portion of the cuff assembly from the interface member.

According to an option, the method may further comprise:
electrically connecting a plug arranged with the cuff assembly into a socket arranged in the control unit housing. Prior to assembling the cuff assembly to the interface member.

According to an option, the interface member is fixed to the control unit housing by screwing.

### BRIEF DESCRIPTION OF THE DRAWINGS

Other features and advantages of the disclosure appear from the following detailed description of one of its embodiments, given by way of non-limiting example, and with reference to the accompanying drawings, in which:

FIG. 1 illustrates a general overview of a monitoring device according to the present disclosure in a use configuration,

FIG. 2 shows a diagrammatic sectional view of the device in place on the left arm of the user, and adjacent to the chest, for the generically defined embodiment,

FIG. 3 illustrates a picture of the device when not in use,

FIG. 4 shows partially the monitoring device illustrating in particular a control unit housing and a resilient cuff holder attached via an interface member,

FIG. 5 shows an exploded view of the monitoring device,

FIG. 6 is a perspective view of an example of the interface member,

FIG. 7 is a perspective view of an example of resilient cuff holder,

FIG. 8 is an enlarged view of the interface area, also illustrating the interlocking function,

FIG. 9 shows a sectional view of the interface member and associated elements,

FIG. 10 shows a functional block diagram.

### DETAILED DESCRIPTION OF THE DISCLOSURE

In the figures, the same references denote identical or similar elements. For clarity purposes, some parts are represented intentionally not at scale with regard to other parts. Also, some parts of timing charts can be represented intentionally not at scale.

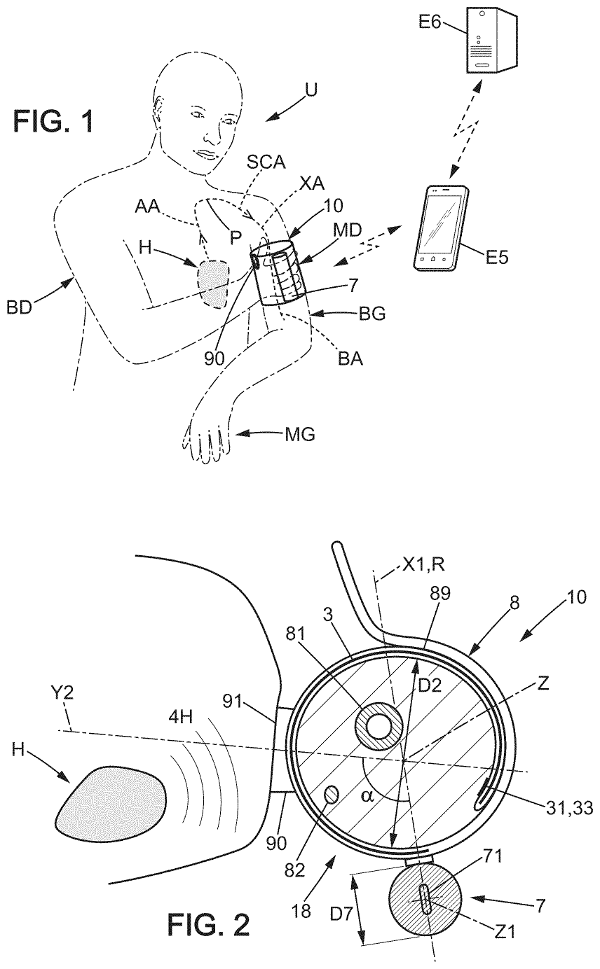

FIG. 1 shows an individual (also ‘user’) U in a configuration where he/she is using a monitoring device according to the present disclosure. The device (otherwise called “apparatus”) is a brachial blood pressure sensing device (named Blood Pressure Monitor i.e. in short “BP Monitor”), the device exhibiting extended functionalities as will be apparent below, so that the device can be called ‘upgraded BP Monitor’.

The user U in question has, among other organs and limbs in his/her anatomy: a left arm BG, a right arm BD, a heart H, a left hand MG a right hand MD.

Further the user in question has an aortic artery (‘aortic arch’) denoted AA, a subclavian artery SCA, an axillary artery XA, a brachial artery BA, belonging generally to the cardiovascular system of the user. Therefore, a blood path of interest noted P is defined as the fluid conduit from the heart H to a reference point at the brachial artery BA.

The device 10 has a wireless communication capability to exchange data with a mobile entity like a smartphone E5 (more generally a mobile device belonging to the user U like a tablet, a laptop . . . ). Such smartphone E5 may in turn exchange data with a remote entity like an Internet server E6 (more generally any resource available somewhere in Internet, not excluding a so-called “cloud” resource).

The monitoring device 10 has either a small display or no display at all, since the user interface capability provided by the smartphone E5 is fully relevant to support displays relating to the use and extended functionalities of the device.

The monitoring device 10 is intended to be used at a home environment, for healthy people as well as people suffering from some disease. It may be used in a medical environment but is particularly suitable to be used by non-medical personnel, i.e. the user under measurement him/herself.

The monitoring device 10 comprises a cuff assembly 18 wrapped around the arm BG, a control unit housing 7. The cuff assembly 18 will be described in more details later. The cuff assembly 18 is coupled to the control unit housing 7 via an interface member 6 whose purpose and details will be given later.

The rest of the time the monitoring device is stowed, notably in a folded configuration as will be seen later.

Cuff Assembly

As illustrated on FIG. 1, the device comprises a cuff assembly (‘armband’ can also be used) wrapped around the arm i.e. the part of the upper limb comprised between the shoulder and the elbow.

In another configuration or variant, the device can be used elsewhere, at the forearm for example, or at the wrist. Generally speaking, the cuff assembly 18 is configured, in use, to surround an upper limb of the user.

As illustrated on FIG. 1, the device is installed on the left arm of the user. However, it is not excluded to use the device elsewhere, at the right upper limb for example.

As illustrated on FIG. 2, the left arm of the user includes a bone named humerus denoted 81, muscles (not especially shown), and the above mentioned brachial artery denoted 82. The humerus extends along an axis denoted Z. The cuff assembly 18, when wrapped around the arm BG, has a general cylindrical shape with a reference axis substantially coinciding with arm axis Z.

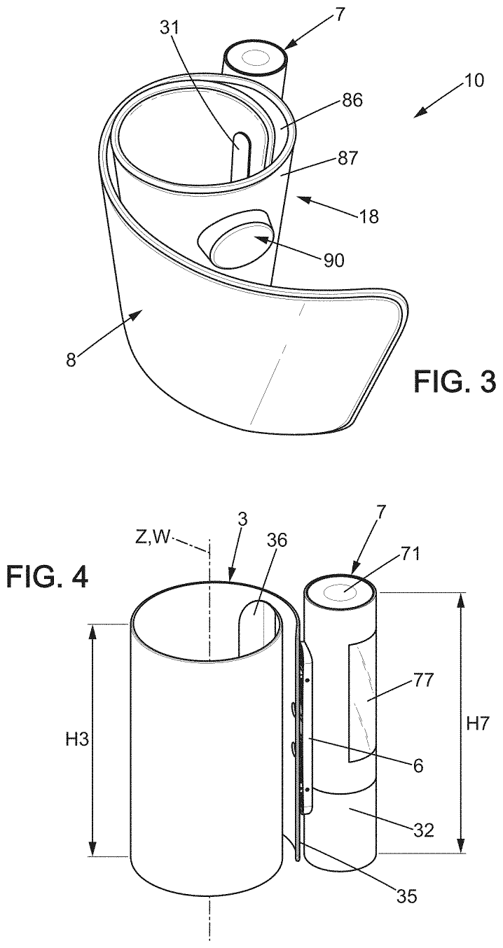

As shown at FIGS. 3 to 5, the cuff assembly 18 comprises, from an innermost item to an outermost item, at least the following components:
an inner layer, denoted 1,
an inflatable bladder, denoted 2,
a cuff holder, denoted 3,
an intermediate sheet, denoted 4,
an outer band, denoted 5.

Regarding overall dimensions of the cuff assembly 18, at flat developed configuration, the length L1 of the cuff assembly along the longitudinal direction L can be comprised between 20 cm and 40 cm. The height H1 along the transverse axis W direction can be comprised between 10 cm and 20 cm.

The inner layer 1 is a thin fabric layer. The inner layer 1 may be made in lycra. Lycra turns out to be a comfortable material, not prone to collect dirt and/or sweat. The inner layer 1 is optional in the frame of the present invention, in particular when the outer wall directed inwardly of the bladder 2 is texturized (likewise textured).

The inflatable bladder 2 is made from two sheets 2A,2B of plastic material (for instance PVC, TPU, PA, etc.) arranged one atop another in a main plane and are welded together at their peripheral border.

The inflatable bladder 2 has two fluid connection ports denoted 99,199. One port is for inflating, deflating, whereas the other one is dedicated to a measuring channel, so that the pressure measured is not perturbed by the unsteady flow of the pump during inflation. In other configurations, there may be only one port, or a single port, for both functions.

The connection ports denoted 99,199 extend perpendicularly to the main plane, along the thickness direction Y of the cuff assembly and along the assembling direction R of interface member 6. Air volume at nominal inflated state can be comprise between 0.05 liter and 0.2 liter.

The cuff holder 3 forms a structural resilient armature of the cuff assembly. The cuff holder is a resilient plastic sheet, with a first longitudinal end 35 attached to the interface member 6 and a second end 36 having a tapered end portion, i.e. with a decreasing thickness to the edge; the first longitudinal end 35 is called proximal and the second end 36 is called distal.

The dimensions of the cuff holder at flat configuration are slightly less than the corresponding dimensions of the bladder.

The thickness of the cuff holder can be comprised between 0.5 mm and 3 mm.

The cuff holder can be made out of polypropylene.

The second end 36 has advantageously a tapered end portion, i.e. a decreasing thickness down to the edge.

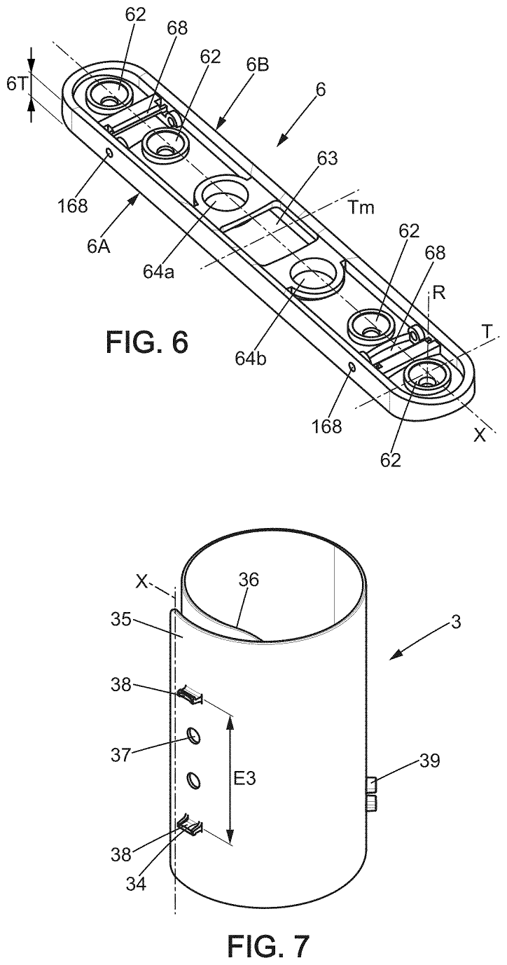

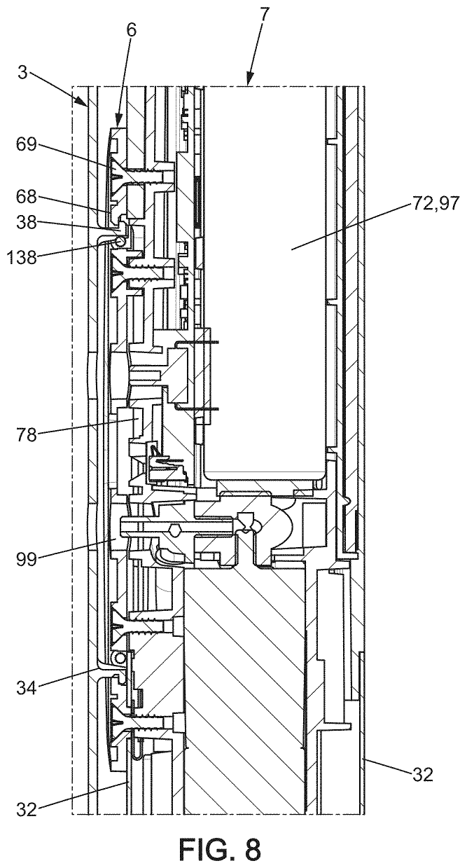

The resilient cuff holder 3 comprises two retaining members 38 at the first longitudinal end 35. In the shown example, the retaining members are formed as hooks, arranged in symmetry with regard to a median plane Tm (cf FIG. 6). The hooks are spaced from one another by a distance E3. E3 is at least 5 cm, or at least 50% of the height H1 of the cuff assembly.

The hook is arranged at an end of a hook arm. In the shown example, there is provided a large footprint for these hook arms, with a rounded shape 34, also named ‘fillet’. The foot/shoe is therefore larger than the hook arm profile itself. It provides a strong implant on the cuff holder sheet.

The resilient cuff holder 3 comprises two through-holes 37, configured to let a suitable passage for the fluid ports 99,199 of the inflatable bladder.

The resilient cuff holder 3 comprises protrusions 39 providing a socket for the attachment of an acoustic sensor 90 and its mechanical securing thereto.

The intermediate sheet 4 is made in an inextensible, i.e. non-stretchable fabric. The intermediate sheet 4 may be made in a nylon fabric.

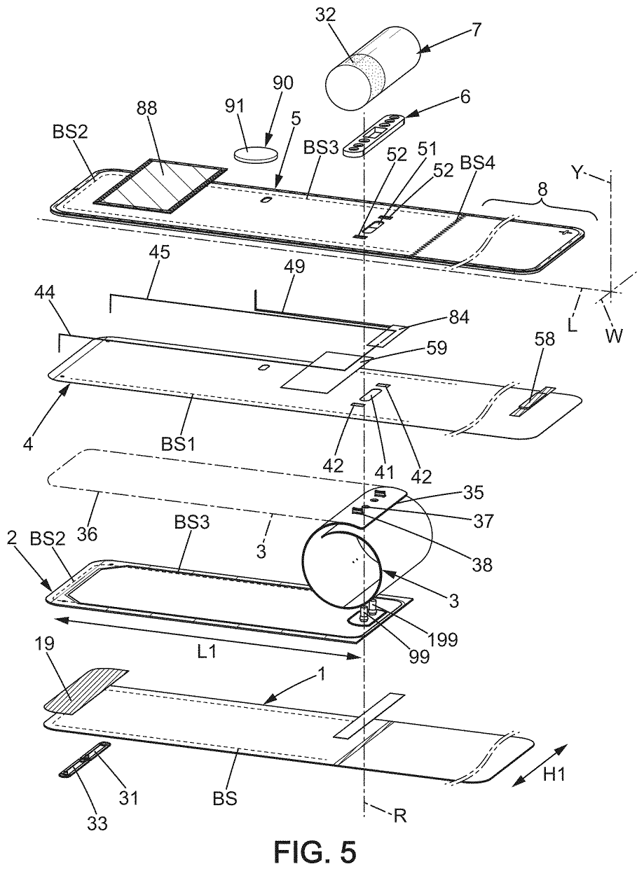

Besides, for the purpose of letting a passage for the one or two fluid ports 99,199 of the inflatable bladder, there is provided a first opening 41 in the intermediate sheet 4 as illustrated at FIG. 5.

Further, the cuff assembly 18 is fixed to the control unit housing, and there are provided second openings 42 in the intermediate sheet 4 to let the passage for at least two hooks of the cuff holder, said hooks being configured to be retained in the control unit housing 7 or an attachment member 6 thereof.

The outer band 5 constitutes the external layer. The outer band 5 can comprise one or more foamed areas. The outer band 5 can comprise fixing means (hooks and loops [i.e. Velcro™] or the like). There may be provided a magnet 58 and a ferrite or metal pad 59 for rollover stowed configuration. The magnet 58 or ferrite 59 can be arranged with the intermediate sheet 4 or with the outer band 5.

The outer band 5 can comprise an extension beyond the cuff assembly forming part or all of an attachment band 8 discussed later.

Besides, for the purpose of letting a passage for the one or two fluid ports 99,199 of the inflatable bladder, there is provided a first opening 51 in the outer band 5 as illustrated at FIG. 5.

Further, the cuff assembly 18 is fixed to the control unit housing, and there are provided second openings 52 in the outer band 5 to let the passage for at least two hooks of the cuff holder, said hooks being configured to be retained in the control unit housing 7 or an attachment member 6 thereof.

Border Joint or Seam

There is provided a border joint at least on three sides on a peripheral border of the inflatable bladder, the border joint joining and securing together the inner layer 1 (optionally), the inflatable bladder 2, the intermediate sheet 4, and the outer band 5.

In the shown example, the border joint is a border seam. There are provided a first border seam BS1, a second border seam BS2, and a third border seam BS3, collectively denoted BS.

There may be provided in fourth border seam BS4 which is made after cuff holder 3 has been inserted inside the cuff assembly between the bladder 2 and the intermediate sheet 4.

According to an alternative solution, instead of making a seam, it is possible to use a technique of ultrasonic or thermal welding.

Control Unit Housing

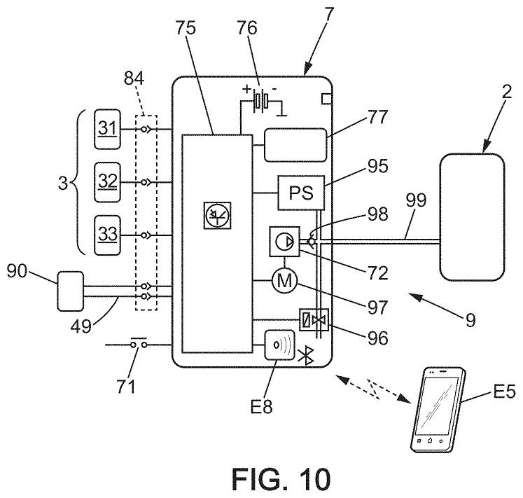

As apparent from FIG. 10, the control unit housing 7 comprises a pneumatic unit 9.

The control unit housing 7 comprises an electronic controller 75 configured to control the pneumatic unit and to determine at least the arterial pressure of the user,

The control unit housing 7 comprises an On/Off switch 71 and a display 77.

The pneumatic unit 9 comprises the already mentioned pump 72 driven by an electrical motor 97, a discharge valve 96, a check valve 98, and a pressure sensor 95.

The control unit housing 7 exhibits generally a cylindrical shape. The diameter D7 of the control unit housing 7 is in some examples less than 40 mm, in some examples around 35 mm. The proposed configuration turns out to be a compact solution which accommodates all the necessary components for the measurement, including a battery 76 for autonomous use.

Interface Member

As already mentioned, with reference to FIG. 6, the assembling direction is denoted R, the elongation axis of the interface member is denoted X, the transverse direction of the interface member is denoted T. Tm designates a median plan parallel to R and T.

According to the illustrated example, the interface member 6 is a separate part interposed between the control unit housing and the cuff assembly for securing them together.

According to another embodiment not shown at the figures, the interface member may be made integral with the control unit housing, for example by using a 3D printing technique.

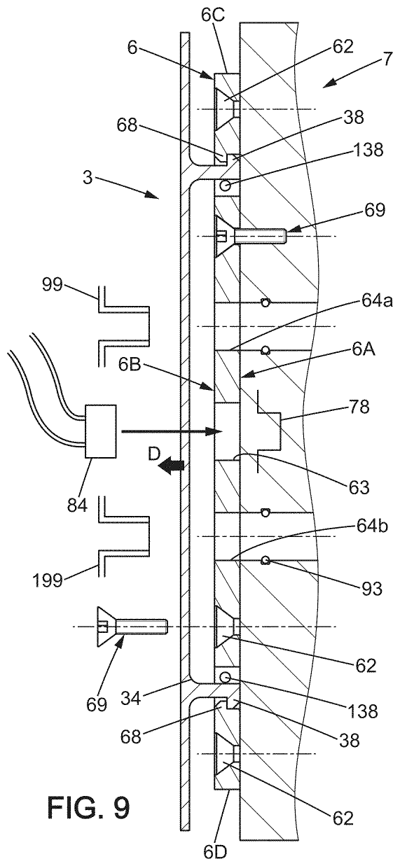

As illustrated at FIGS. 6 and 9, the interface member 6 is screwed to the control unit housing. More precisely, there are provided through holes 62 in the interface member.

Screws 69 are provided to fix the interface member to the control unit housing.

In the shown example, the through holes 62 are frustoconical thru bore, and the proposed screws are of the type countersunk head screw.

There is provided in a middle portion of the interface member at least a through hole 63 for electrical wire connection/coupling, two through holes 64a,64b for pneumatic fluid piping/coupling.

There is provided threaded holes in the body of the control unit housing 7 for receiving the screws 69. There may be provided 2 or 4 screws in the attachment (4 illustrated).

The interface member 6 comprises two complementary retaining members 68, configured to be snap-fit with the retaining members 38. Here the complementary retaining members are in the form of shoulders or elbows, i.e. stop abutments preventing retaining members from moving away from the control unit housing.

Advantageously, the free extremities of the retaining members 38 are directed towards the edges of the cuff assembly, i.e. away from each other. Thanks to the resilience of the cuff-holder 3, this system makes it easy to disassemble the cuff assembly from the control unit housing 7 by applying an inward pressure (arrow D at FIG. 9) on the cuff assembly (in the R-direction) near the intermediate portion of the interface member 6. Assembly and disassembly require no additional tool or machinery, are very fast, and can thus be repeated at will, which is very convenient for testing and changing parts of the device.

In one embodiment, the interface member 6 comprises transverse holes 168 arranged in the transverse direction (T) for receiving the interlock pins 138. The interlock pins prevent disassembly of complementary retaining members 68 versus retaining members 38. Said interlock pins 138 and transverse holes 168 have in some examples a diameter less than 2 mm, therefore, this is nearly hidden and difficult to tamper.

Instead of interlock pins, any other mechanical parts having an interlocking function can be used like thin rod-like screw with slotted head. Therefore, the clause “interlock element” is generically used.

We note that interlock elements 138 can easily be removed, making possible and easy to disassemble the cuff-assembly from the control unit housing 7. We note here that the hooks 68 are oriented away from each other.

The interface member 6 exhibits a first face 6A oriented towards the control unit housing 7 and a second face 6B oriented towards the cuff assembly.

The first face 6A is concave and is configured to follow the cylindrical body of the control unit housing. The second face 6B is substantially flat or exhibits a slight concavity.

The longitudinal ends of the interface member denoted respectively 6C and 6D. The distance between the two ends 6C,6D is at least 10 cm or two-thirds of the height H1 of the cuff-holder.

There is provided a connector 84 attached to connection wires discussed later. In one example, the connector is a flat flex PCB-type connector. The connector is inserted through the central through hole 63, and is plugged onto a corresponding counterpart electrical socket 78 provided in the control unit housing.

Regarding the pneumatic connections, there may be provided O-rings 93 in the control unit housing or likewise complementary tubes, such that the connection ports 99,199 are received in an airtight manner into pneumatic components of the control unit housing.

It is also apparent from FIG. 9 that disassembling the cuff assembly from the interface member is possible provided that the interlock members are not yet inserted. More precisely, if a pressing effort is exerted along the arrow D, the hooks tend to move a bit toward each other therefore enabling disassembly of the hooks from the retaining shoulders.

In some examples, the snap fitting mechanism is tight fitted and coming flush in contact with the housing surface. A tight fit should be understood as a dimensioning preventing movement of the different pieces once in place, in particular along a direction perpendicular to a plane defined by an external surface of the housing. This not only permits obtaining a firm structure, but also avoids including orifices or recesses in the housing which would otherwise be included in order to permit lodging parts of retaining members which would extend into the housing. In the example illustrated in FIG. 9, whereby the snap fitting mechanism comprises the retaining members 38, the retaining members 38 are tight fitted between, on one hand, housing 7, and, on the other hand, interface member 6. In this example, each retaining member 38 abuts flush against an external surface of the housing 7, the external surface of the housing 7 being convex and continuous. In this example, each retaining member also abuts flush against a corresponding surface of the interface member, the corresponding surface pertaining to the complementary retaining members 68. In other words, each retaining member 38 is firmly caught between a complementary retaining member 68 of the interface member and a convex continuous outside or external surface of the housing 7.

Attachment Band

There is provided an attachment band 8. The attachment band 8 extends from the cuff assembly along a longitudinal direction L configured to momentarily secure the attachment band to the outer band of the cuff assembly during a measurement cycle.

The attachment band 8 is flexible and comprises fixing means for securing that number to the external wall 87 of the cuff assembly 18 at least during the blood pressure measurement. The proper position is chosen by the user according the size of his/her arm.

In one configuration, the fixing means may comprise loop and hook pads, a loop pad 88 at one location and a hook pad 89 at another counterpart location, such that adjustment and securing of various encompassed circumferences (πD2) of user's arm is made available. This allows convenient attachment whatever the size of the arm.

Sensors (Stetho and ECG)

The device 10 may comprise additionally an acoustic sensor denoted 90.

The cuff assembly 18 has an internal wall denoted 86 intended to contact the arm's skin (or light clothing) and to press against the arm. The armband has an external wall denoted 87 on the opposite side of the band with regard to the internal wall 86.

In use configuration, the acoustic sensor 90 is located against the chest, i.e. the left side of chest. Sound waves 4H emitted by the heart are sensed by a sensitive portion 91 of the acoustic sensor 90 the sensitive portion 91 bearing on the left-side chest, i.e. adjacent to the chest. Acoustic waves 4H are converted into electrical signals per se thus not detailed here. It should be noted that acoustic waves 4H can be sensed without trouble through a light clothing, an underwear or the like.

According to one particular option, the device is further equipped with an ECG function, i.e. ElectroCardioGraphic function.

For this purpose, there are provided three contact electrodes 31, 32, 33, the three of them integrated in the device, without the need to have linking wires like in most prior art devices.

The first electrode 31 is arranged on the internal wall 86 of the band and has a sensitive face oriented toward the skin of the arm. The third electrode 33 is also arranged on the internal wall 86 and has also a sensitive face oriented toward the skin of the arm.

Each of the electrodes is formed as a thin pad of a surface comprised between 2 cm² and 10 cm²; a surface between 5 cm² and 7 cm² can be chosen; the shape of the thin pad may be somewhat curved to follow the standard curvature of the skin of the arm.

The first and third electrodes 31,33 are arranged at a cuff assembly distal end (second end 36) opposed to the interface member for example one aside the other transversally (along W). Alternatively, first and third contact electrodes 31, 33 can be arranged differently, for example one aside the other along L. In the shown example, there is provided a back pad 19 to support the contact electrodes 31, 33. The Lycra inner layer 1 is sandwiched at this place between the back pad 19 and the contact electrodes 31, 33.

Whenever the cuff assembly is pressurized, first and third contact electrodes 31, 33 are firmly pressed against the skin of the arm, thereby ensuring a fairly good contact with a small electrical contact resistance. It should be noted that no gel is required at the contact electrode contrary to prior art habits. Contact electrodes are to be placed against the bare skin; however, thanks to the pressure, it is not excluded to have a light underwear cloth between the skin and the electrodes.

The contact electrodes can be made of stainless steel, silver, or other coated materials (coated with silver, chromium, gold, titanium or platinum), not excluding materials coated by physical vapor deposition technique (PVD techniques). The contact electrodes can also be made of a conducting wire (stainless steel, silver, etc.) sewed to the inner layer.

It is to be noted that two electrodes might be sufficient, therefore the third electrode 33 is considered optional.

Regarding the second electrode 32, it is arranged around the external surface of the control unit assembly as best seen at FIG. 4. A conductive material forms a coating of at least a part of the control unit housing. A metallic coating material (silver, titanium, chromium), are deposited by physical vapor deposition technique (PVD techniques). It can also be an empty cylinder made out of the conductive material.

In one example, the second electrode covers the lower third of the cylinder, for example all around the accessible circumference by the fingers of the user (see FIG. 1). Therefore, it is easy for the user to grab/seize the second electrode with a good electrical contact. According to one example, the second electrode lies over the 20% lower part of the control assembly unit; in other implementation, its height can be bigger, like 30% or 40%.

The device further comprises connection wires to provide electrically coupling between sensors and the control unit housing 7. Connection wires 44,45 electrically couple the contact electrodes with the control unit housing.

Connection wires 49 are provided to electrically couple the acoustic sensor with the control unit housing. The connection wires are integrated into the cuff assembly and they are protected therein and not visible from the outside. As depicted at FIG. 5, since the connection wires are arranged at an outer zone of the bladder, in some examples between the intermediate sheet (4), and the outer band (5), they do not hinder inflation and measurement.

The wires 44,45,49 are electrically coupled to the above mentioned connector 84.

Overall System

The control unit housing 7, the attachment band 8 and the cuff assembly have substantially the same height (H1,H3,H7) along the transverse axis W (coincides with elongation axis X for the interface member). This provides an aesthetic assembly and eases rollup configuration. This configuration allows optimization of the occupied volume.

As shown at FIG. 2, the control unit housing 7 and the acoustic sensor 90 are arranged at an angle α comprised between 90° and 140° with regard to the cylindrical configuration of the cuff assembly, as defined in a use configuration on a 30 cm reference arm circumference.

As shown at FIG. 10, there is provided a wireless coupling E8 with smartphone E5.

It is to be noted that the switch 71 can be physical switch, or tap switch or a tactile area.

It shall be understood that the use of interlock member(s) is not compulsory. In one embodiment, there may be provided strong snap-fit connection retaining members and complementary retaining members. There can be provided two or more hooks and complementary shoulders or stop abutments. The various hooks can exhibit different orientations so that mechanical torques along different orientation can be with withstood without disassembly.

1. A cuff-type monitoring device, for collecting cardiovascular data relating to an individual user, comprising:
a control unit housing including at least a pump,
a cuff assembly including a resilient cuff holder and configured, in use, to surround an upper limb of the individual, and
an interface member arranged at the control unit housing, provided as a separate part from the control unit housing,
wherein there is provided at least a pneumatic coupling and at least an electrical coupling through the interface member, for coupling the cuff assembly to the control unit housing,
wherein the resilient cuff holder comprises two retaining members,
the interface member comprising two complementary retaining members, configured to be snap-fit with the retaining members.

2. The device according to claim 1, wherein the interface member is a separate part interposed between the control unit housing and the cuff assembly for securing them together.

3. The device according to claim 2, wherein the interface member is screwed to the control unit housing.

4. The device according to claim 1, wherein the retaining members are formed as hooks.

5. The device according to claim 4, wherein two hooks are provided spaced from one another of a distance at least 5 cm, or at least 50% of the height of the cuff assembly.

6. The device according to claim 1, wherein interlock elements/pins in holes along the transverse direction are provided, and wherein the interface member comprises transverse holes arranged in the transverse direction for receiving the interlock pins.

7. The device according to claim 1, wherein the interface member a first face oriented towards control unit housing and a second face oriented towards the cuff assembly, wherein the first face is concave and is configured to follow the cylindrical body of the control unit housing.

8. The device according to claim 1, wherein the cuff holder a resilient plastic part with retaining members or hooks integrally formed therein.

9. The device according to claim 1, wherein the interface member is an elongated flat plastic part.

10. The device according to claim 9, wherein the elongated flat plastic part has a thickness along an assembling direction of less than 6 mm.

11. The device according claim 1, wherein at least a through hole is provided in a middle portion of the interface member for electrical wire connection, and at least one through further holes is provided for pneumatic fluid piping.

12. The device according to claim 1, wherein an ECG electrodes is disposed around at least a part of the control unit housing and the interface member is configured to securely fix the ECG electrode on the control unit housing.

13. The device according to claim 1, wherein the interface member is made integral with the control unit housing.

14. The device according to claim 1, whereby the two complementary retaining members configured to be snap-fit with the retaining members form a tight fit.

15. The device according to claim 14, whereby the retaining members are configured to be flush in contact with a housing surface.

16. An assembly method for assembling a cuff-type monitoring device, for collecting cardiovascular data relating to an individual user, the method comprising:
providing a control unit housing including at least a pump;
providing a cuff assembly including a resilient cuff holder;
providing an interface member;
fixing the interface member to the control unit housing;
assembling the cuff assembly to the interface member, by inserting two retaining members into two complementary retaining members; and
Securing the cuff assembly to the interface member by inserting interlock elements.

17. The assembly method according to claim 16, further comprising:
electrically connecting a plug arranged with the cuff assembly into a socket arranged in the control unit housing, prior to assembling the cuff assembly to the interface member.

18. The assembly method according to claim 16, wherein the interface member is fixed to the control unit housing by screwing.

19. The assembly method according to claim 16, wherein the assembling the cuff assembly to the interface member, by inserting two retaining members into two complementary retaining members comprises a tight fitting assembly.

20. The assembly method according to claim 19, wherein the assembling the cuff assembly to the interface member, by inserting two retaining members into two complementary retaining members comprises the retaining members coming flush in contact with a housing surface.
